(19) United States
(12) Patent Application Publication
(10) Pub. No.: US 2017/0021173 A1
(43) Pub. Date: Jan. 26, 2017

(54) SYSTEM AND METHOD FOR MEDICAL DEVICES AND PAIN REDUCTION

(71) Applicant: ANDREAS PENEDER, San Carlos, CA (US)

(72) Inventor: ANDREAS PENEDER, San Carlos, CA (US)

(21) Appl. No.: 15/216,608

(22) Filed: Jul. 21, 2016

Related U.S. Application Data
(60) Provisional application No. 62/195,222, filed on Jul. 21, 2015.

(51) Int. Cl. A61N 1/36; A61N 1/04

(52) U.S. Cl. CPC A61N 1/36021; A61N 1/0456

(57) ABSTRACT

A method and system providing pain relief by applying electromagnetic impulses to nerves which transport the pain signals from the areas effected by pain to the correlated pain receptors in the brain. An array of two or more topical sensors, applied above the pain-conducting nerve read the electro-magnetic waves emitted by the respective nerve duct; one or more pulse generators emit an electromagnetic wave designed to cancel the pain signal by ways of destructive interference.

### CROSS-REFERENCE TO RELATED APPLICATIONS

This application claims priority to U.S. provisional patent application Ser. No. 62/195,222, filed Jul. 21, 2015. Priority to the provisional patent application is expressly claimed, and the disclosure of the provisional application is hereby incorporated herein by reference in its entirety and for all purposes.

### FIELD

The disclosed embodiments relate generally to medical devices and more specifically, but not exclusively, to systems and methods for reducing pain by applied neuromodulation to emit electromagnetic impulses antipodal to a pain signal.

### BACKGROUND

Pain travels as a chemo-electrical signal through the human body. The chemo-electrical signal uses the same nerve-tracts that transport information about temperature, pressure, and/or touch and relays commands regarding motion and contraction or relaxation of muscles. The speed at which nerve signals travel through the nerve-tracts has a maximum velocity of about one hundred twenty meters/second. For example, the average nerve has thermos-receptors registering warmth at about two meters/second. For the purposes of this disclosure, the nerve signal can be considered an electromagnetic wave.

Application of electricity for pain treatment dates back thousands of years. Ancient Egyptians and later the Greeks and Romans recognized that electric fish are capable of generating electric shocks for relief of pain. In the eighteenth and nineteenth centuries, these natural producers of electricity were replaced by man-made electric devices. The nineteenth century was the “golden age” of electrotherapy—being used for countless dental, neurological, psychiatric and gynecological disturbances.

The pain relieving action of electricity was explained in particular by two main mechanisms: first, segmental inhibition of pain signals to the brain in the dorsal horn of the spinal cord and second, activation of the descending inhibitory pathway with enhanced release of endogenous opioids and other neurochemical compounds (e.g., serotonin, noradrenaline, gamma aminobutyric acid (GABA), acetylcholine and adenosine).

Modern electrotherapy of neuromuscular-skeletal pain is based in particular on the following types: transcutaneous electrical nerve stimulation (TENS), percutaneous electrical nerve stimulation (PENS or electro-acupuncture) and spinal cord stimulation (SCS). In mild to moderate pain; TENS and PENS are somewhat effective methods, whereas SCS is useful for therapy of refractory neuropathic or ischemic pain. In 2005, high tone external muscle stimulation (HTEMS) was introduced. In diabetic peripheral neuropathy, its analgesic action was more pronounced than TENS application. HTEMS appeared also to have value in the therapy of symptomatic peripheral neuropathy in end-stage renal disease (ESRD). Besides its pain-relieving effect, electrical stimulation is of major importance for prevention or treatment of muscle dysfunction and sarcopenia. In controlled clinical studies electrical myostimulation (EMS) has been shown to be effective against the sarcopenia of patients with chronic congestive heart disease, diabetes, chronic obstructive pulmonary disease and ESRD.

In other words, modern electrotherapy of neuromuscular-skeletal pain emit random electric impulses to the pain afflicted areas. Accordingly, these conventional systems only are effective for a very limited number of patients. In fact, efficacy is statistically significant for all of the above methods; but in clinical studies still only 12-15% of pain patients report an effective reduction in pain levels when treated with TENS devices.

In view of the foregoing, a need exists for an improved system for electrical stimulation-based pain reduction in an effort to overcome the aforementioned obstacles and deficiencies of conventional medical systems.

### SUMMARY

The present disclosure relates to a system and method for pain reduction based on electrical stimulation. The pain reduction system and method represents a new paradigm of pain management. The present system not only provides pain patients with a significantly higher pain reducing efficacy than prior systems, but also enables patients to easily treat pain symptoms without undergoing invasive procedures. A user-friendly mobile application, which is connected with the pain reduction system (e.g., wired or wirelessly), allow the patients to efficiently position sensors of the pain reduction system on top of the nerve conducting the targeted pain signal. Furthermore, the mobile application provides functionality to easily identify the pain signal amidst the plethora of sensory and motoric signal transmitted through a nerve duct at any given moment; thus eliminating unwanted side-effects like tremors or numbness

### BRIEF DESCRIPTION OF THE DRAWINGS

FIG. 1 is an exemplary top-level block diagram illustrating one embodiment of a pain reduction system cooperating with a nerve cell.

FIG. 2 is an exemplary diagram illustrating one embodiment of an axon with a series of sequential action potential that can be used with the pain reduction system of FIG. 1.

FIG. 3 is an exemplary top-level block diagram illustrating an embodiment of the pain reduction system of FIG. 1.

FIG. 4 is an exemplary diagram illustrating another embodiment of the pain reduction system of FIG. 1.

FIG. 5 is an exemplary diagram illustrating another embodiment of the pain reduction system of FIG. 1.

FIG. 6 is an exemplary diagram illustrating an embodiment of the pain reduction system of FIG. 5.

FIG. 7 is an exemplary diagram illustrating another embodiment of the pain reduction system of FIG. 1.

FIG. 8A is exemplary graph diagram illustrating the one embodiment of constructive and destructive interference principles used by the pain reduction system of FIG. 1.

FIG. 8B is an exemplary graph diagram illustrating one embodiment of an electromagnetic waveform representing a pain signal that can be detected by the pain reduction system of FIG. 1.

FIG. 8C is an exemplary graph diagram illustrating one embodiment of an electromagnetic waveform representing a corresponding cancellation signal generated by the pain reduction of FIG. 1 for the pain signal shown in FIG. 8B.

FIG. 8D is an expanded view of the pain signal of FIG. 8B illustrating one embodiment of a bandwidth of signals for elimination by the pain reduction system of FIG. 1.

FIG. 9 is an exemplary flow diagram illustrating one embodiment of a method for pain reduction using the pain reduction system of FIG. 1.

FIG. 10 is an exemplary flow diagram illustrating one embodiment for positioning the topical sensors of the method for pain reduction of FIG. 9.

FIG. 11 is an exemplary flow diagram illustrating another embodiment for positioning the topical sensors of the method for pain reduction of FIG. 9.

FIG. 12 is an exemplary flow diagram illustrating one embodiment for generating the cancellation signal of the method for pain reduction of FIG. 9.

FIG. 13 is an exemplary flow diagram illustrating another embodiment for generating the cancellation signal of the method for pain reduction of FIG. 9.

FIG. 14 is an exemplary flow diagram illustrating another embodiment for generating the cancellation signal of the method for pain reduction of FIG. 9.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

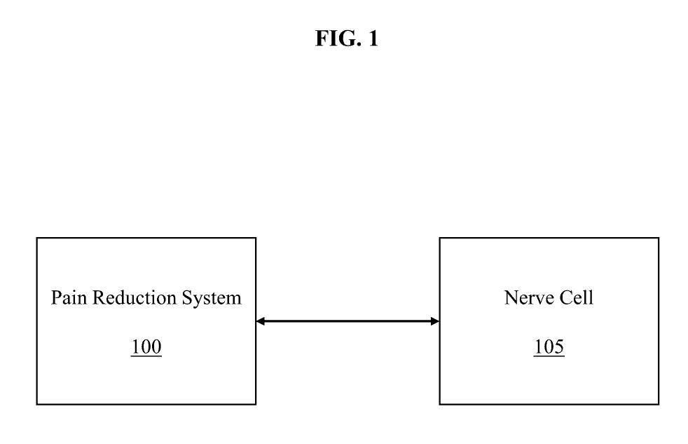

Since currently-available electric nerve modulation-based pain reduction systems are deficient because they emit random electric impulses to the pain afflicted areas and are only effective for a very limited number of patients, an improved system for pain reduction can prove desirable and provide a basis for a wide range of medical applications, such as providing pain relief for patients with chronic diabetic neuropathy, patients with polyneuropathy, or patients with phantom pain after amputation of peripheral extremities. This result can be achieved, according to one embodiment disclosed herein, by a pain reduction system 100 as illustrated in FIG. 1.

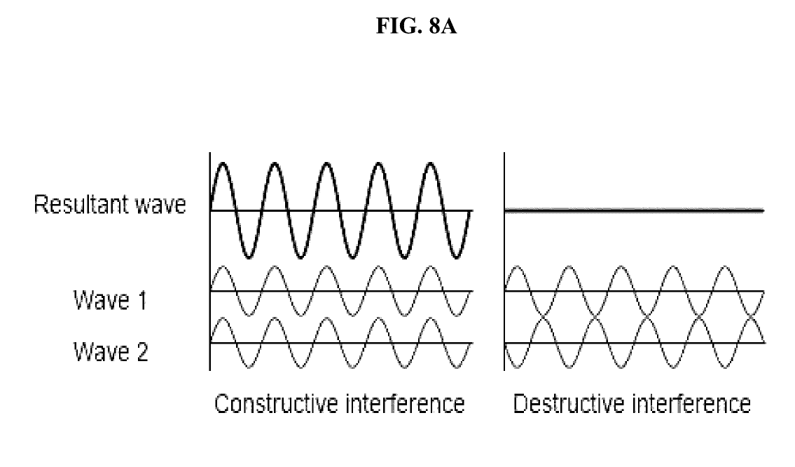

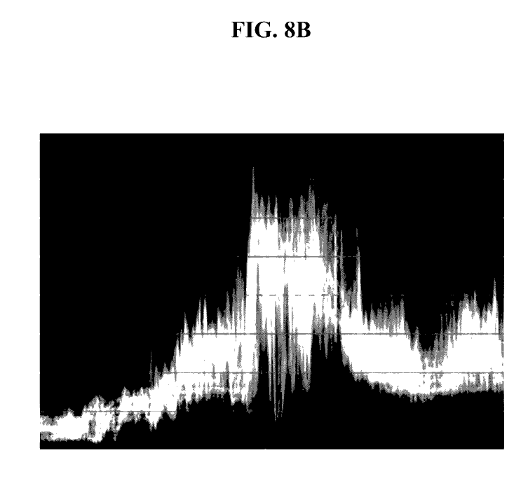

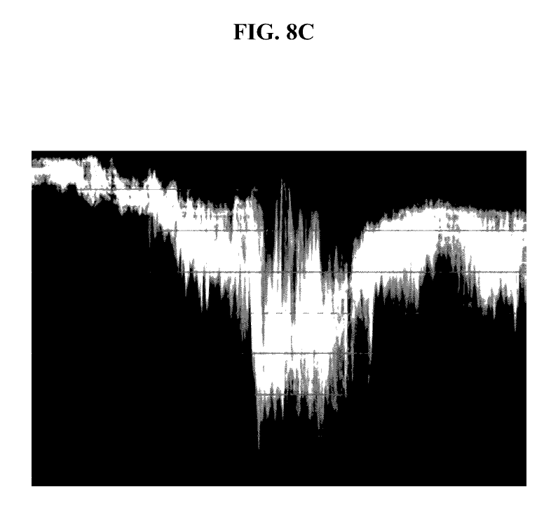

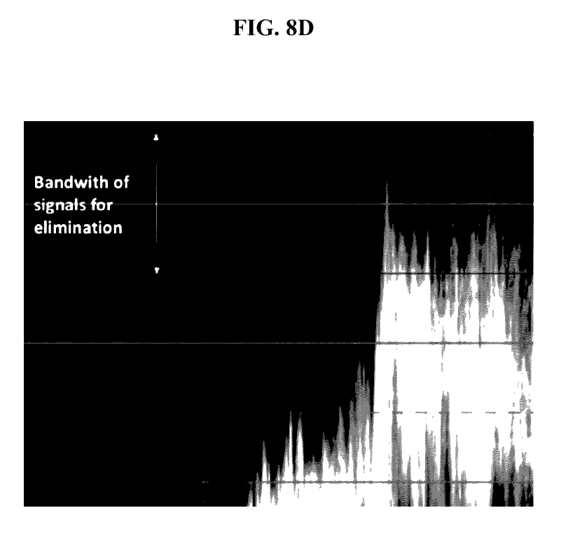

Turning to FIG. 1, the pain reduction system 100 analyzes the waveform of a nerve signal (e.g., shown in FIG. 8) produced by a nerve cell 105, then generates a signal that will invert the polarity of the original signal to provide a reduction in pain.

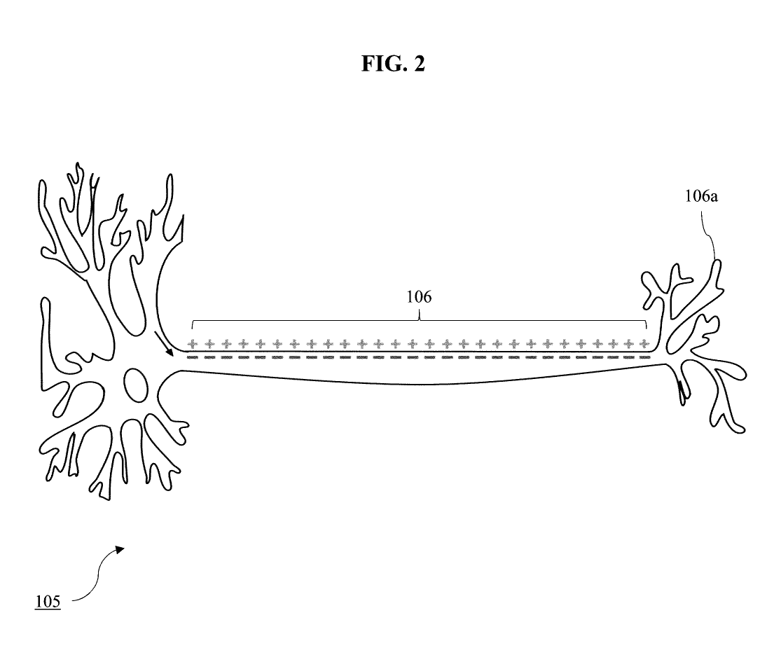

With reference to FIG. 2, nerve impulses (or spikes) travel along axons 106. An axon 106 is a long, slender projection of a nerve cell 105, or neuron, that typically conducts electric impulses away from the neuron's cell body. The nerve impulses travel from one nerve cell 105 to the next via a sequence of short-lasting events in which the electric membrane potential of a nerve cell 105 rapidly rises and falls, following a consistent trajectory.

As an action potential travels down the axon 106, there is a change in polarity across the membrane. The Na+ and K+ gated ion channels open and close as the membrane reaches the threshold potential, in response to a signal from another nerve cell 105. At the beginning of the action potential, the Na+ channels open and Na+ moves into the axon 106, causing depolarization. Repolarization occurs when the K+ channels open and K+ moves out of the axon 106. This creates a change in polarity between the outside of the cell and the inside. The impulse travels down the axon in one direction only, to an axon terminal 106a where the axon terminal 106a signals other nerve cells 105.

While the physical foundation of this process is fundamentally different from the way an electromagnetic wave travels along, for example, a copper wire, the behavior and properties of the resulting signals are quite similar. So similar, in fact, that the same set of differential equations used by Fourier to describe heat conduction in a wire are still in use to establish mathematical theories of nerve fiber conduction and for modeling the behavior of axons 106.

In short and radically simplified: a nerve signal is an electromagnetic wave that travels through the body along conducts (i.e., nerves).

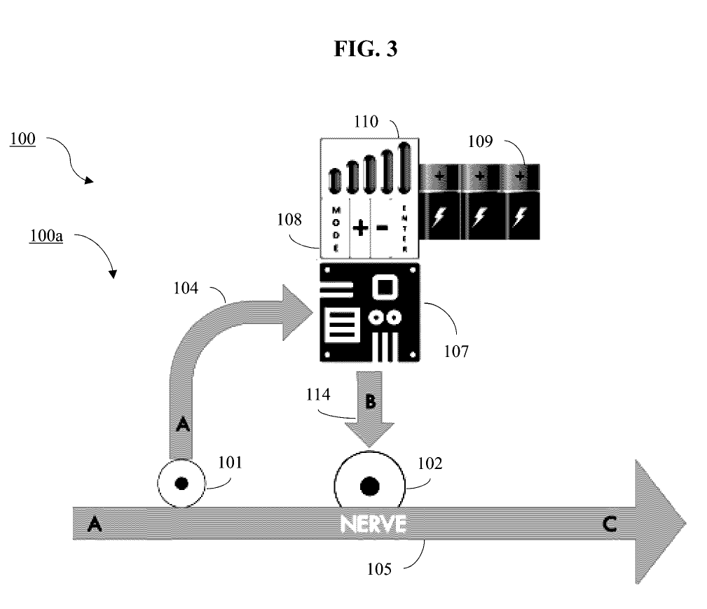

One embodiment of the pain reduction system 100 is shown in FIG. 3. Turning now to FIG. 3, a pain reduction system 100a includes a topical sensor 101 which reads a pain signal A traveling through the nerve cell 105 and transmits the signal A to a reLeaph processing unit (rPU) 107 via a connector 104. In order for the sensor 101 to successfully read the pain signal A, the sensor 101 can be positioned right on top of the nerve 105. As shown, the nerve 105 is illustrated as an arrow for exemplary purposes only to indicate a direction that the pain signal A travels through the nerve cell 105. However, it should be understand that electromagnetic waves can travel through the body via the nerve 105 in any direction. In some embodiments, the sensor 101 can include one or more electrodes to measure and receive electrical signals through the body.

In some embodiments, the pain reduction system 100a can operate in a positioning mode via a selector button 108 and the sensor 101 can be positioned across the skin of the patient in roughly the vicinity of the nerve 105. The pain reduction system 100a includes a display 110 to show the nerve signal strength and allow the sensor 101 to be positioned where the highest signal level is shown. The display 110 can use up a significant amount of electricity; a battery pack 109 can be sizeable in order to power both the display 110 and the rPU 107.

The rPU 107 creates an opposing cancellation signal B and transmits this signal via a connector 114 to a pulse generator 102. The pulse generator 102 is positioned topically and close to the sensor 101 (e.g. farther away from the pain afflicted area and closer to the pain receptors than the placement of the sensor 101). In a preferred embodiment, the pain signal A, still traveling along the nerve 105, and the cancellation signal B cancel each other out once they collide, which will be discussed with reference to FIG. 8.

In this embodiment, the patient can calibrate for nerve velocity manually. Stated in another way, the patient can account for any timing that is required for the pain signal A to be received by the rPU 107 in order to provide the cancellation signal B at an appropriate time. The patient selects the appropriate device mode (i.e., via the selector button 108) and delays and/or advances the timing of the cancellation signal B via modification buttons (shown in FIG. 3 as + and −buttons) and the selector button 108. Furthermore, fat and muscle tissue can distort and weaken the signals A and B. Therefore, the collision of signals initially may not result in a perfect cancellation, but in a reduction of pain intensity. In this embodiment, the patient also can calibrate for distorted and weakened signals manually. The patient can select the appropriate device mode (i.e., via the selector button 108) and change the signal intensity (via the buttons + and − and mode selector button 108). With successful calibration, the collision of the original pain signal A and the cancellation signal B results in a cancelled pain signal and a patient free of pain.

Additionally and/or alternatively, the pain reduction system 100 can be water-resistant. Accordingly, the patient can eliminate chronic pain symptoms and take a bath or shower without having to remove any portion of the pain reduction system 100.

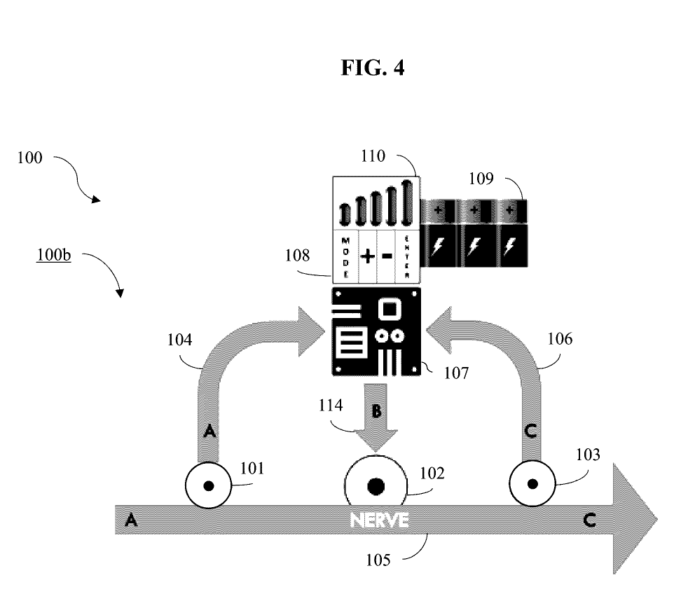

Turning to FIG. 4, another embodiment of the pain reduction system 100 is shown. A pain reduction system 100b includes the topical sensor 101 which reads the pain signal A traveling through the nerve cell 105 and transmits the signal A to the reLeaph processing unit (rPU) 107 via the connector 104. The pain reduction system 100b also includes a test sensor 103 for receiving a collision signal C (i.e., signals A+B) traveling through the nerve cell 105 and transmits the collision signal C to the reLeaph processing unit (rPU) 107 via connector 106. In order for the sensors 101 and 103 to successfully read the pain signal A and the collision signal C, respectively, the sensors 101 and 103 can be positioned right on top of the nerve 105. In some embodiments, similar to the sensor 101, the test sensor 103 can include one or more electrodes to measure and receive electrical signals through the body.

In some embodiments, the pain reduction system 100b can operate in the positioning mode via the selector button 108 and the sensors 101 and 103 can be positioned across the skin of the patient in roughly the estimated position (e.g., within 4 square centimeters) of the nerve 105. The pain reduction system 100b includes the display 110 to show the nerve signal strength and allow the sensors 101 and 103 to be positioned where the highest signal level is shown. The battery pack 109 can be sizeable in order to power both the display 110 and the rPU 107.

The rPU 107 creates an opposing cancellation signal B and transmits this signal via a connector 114 to the pulse generator 102. The pulse generator 102 is positioned topically and close to the sensor 101 (e.g. farther away from the pain afflicted area and closer to the pain receptors than the placement of the sensor 101 but positioned between the sensor 101 and the sensor 103). In a preferred embodiment, the pain signal A, still traveling along the nerve 105, and the cancellation signal B cancel each other out once they collide.

In this embodiment, the rPU 107 calibrates for nerve velocity automatically. In automatic calibration mode, the rPU 107 emits a test signal through the pulse generator 102, measures the time until this test signal reaches the sensors 101, 103 and determines pertaining nerve velocity. According to the determined pertaining nerve velocity, the rPU 107 delays and/or advances the cancellation signal B by a predetermined time in order to match it with the initial pain signal A.

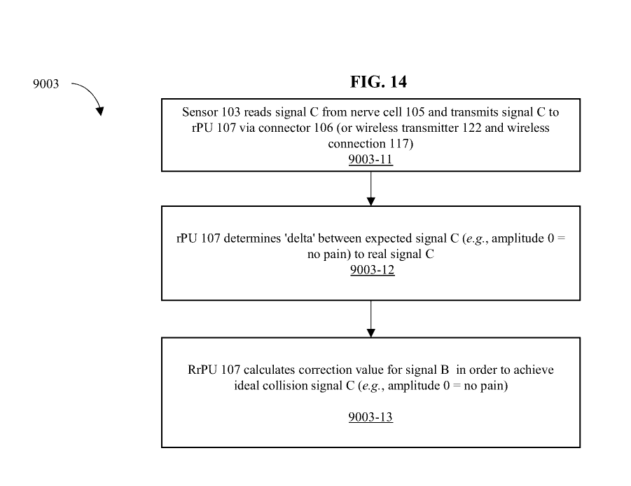

In some embodiments, the rPU 107 can automatically calibrate for weakened and distorted signals, such as explained with reference to FIG. 14.

The sensor 103, positioned near the pulse generator 102 (e.g., about three centimeters from the pulse generator 102 and farther away from the pain afflicted area and closer to the pain receptors than the pulse generator 102), reads the resulting collision signal C and sends it back to the rPU 107 via a connector 106. The rPU 107 determines a delta (Δ) between an optimum collision result and the actual collision signal C and corrects subsequent cancellation signals B for Δ in order to converge to the optimum collision result: a cancelled pain signal and a patient free of pain.

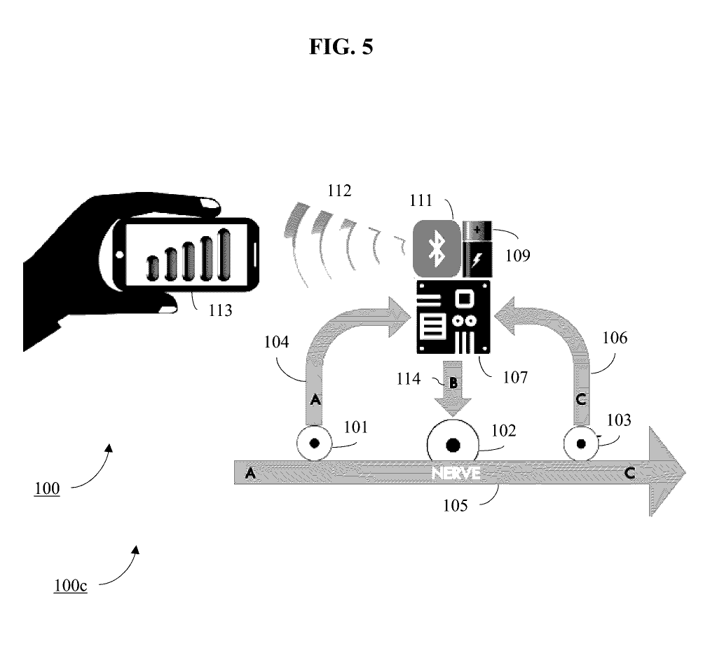

Another embodiment of the pain reduction system 100 is shown in FIG. 5. Turning now to FIG. 5, a pain reduction system 100c includes the topical sensor 101 which reads the pain signal A traveling through the nerve 105 and transmits this signal to the reLeaph processing unit (rPU) 107 via the connector 104. The pain reduction system 100b also includes the test sensor 103 for receiving the collision signal C and transmits the collision signal C to the reLeaph processing unit (rPU) 107 via connector 106. In order for the sensors 101 and 103 to successfully read nerve signal A and the collision signal C, respectively, the sensors 101 and 103 can be positioned right on top of the nerve 105.

In some embodiments, the pain reduction system 100c can operate in a positioning mode via a mobile application on a mobile device 113 (e.g., smartphone) which is connected via a wireless connection 112 (e.g., such as via Bluetooth) and a wireless transmitter 111. As shown, the mobile device 113 is connected via a wireless connection 112, thereby reducing the number of wires and maintaining a convenient handling by a user; however, it should be understood that a wired connection also is within the scope of this embodiment. The sensor 101 can be moved across the skin in roughly the vicinity of the nerve 105. The mobile device 113 shows the nerve signal strength and the pain reduction system 100c can be positioned such that the sensors 101 and 103 where the highest signal level is shown.

The rPU 107 creates an opposing cancellation signal B and transmits this signal via the connector 114 to the pulse generator 102. This pulse generator 102 is positioned topically and close to the sensor 101 (e.g., farther away from the pain afflicted area and closer to the pain receptors than the position of the sensor 101). In a preferred embodiment, the original pain signal A, still traveling along the nerve 105, and the cancellation signal B cancel each other out once they collide.

In this embodiment, the rPU 107 calibrates for nerve velocity automatically. In an automatic calibration mode, the rPU 107 emits a test signal through the pulse generator 102, measures the time until this test signal reaches the sensors 101, 103 and determines pertaining nerve velocity. According to this value, the rPU 107 delays and/or advances the cancellation signal B by a predetermined time to match it with the initial pain signal A.

In some embodiments, the rPU 107 can automatically calibrate for weakened and distorted signals, such as explained with reference to FIG. 14.

The sensor 103, positioned near (e.g., within 3 centimeters of) the pulse generator 102 (e.g., farther away from the pain afflicted area and closer to the pain receptors than the pulse generator 102), reads the resulting collision signal C and sends it back to the rPU 107 via a connector 106. The rPU 107 determines a delta (Δ) between an optimum collision result and the actual collision signal C and corrects subsequent cancellation signals B for Δ in order to converge to the optimum collision result: a cancelled pain signal and a patient free of pain.

As used herein, the signal A has been referred to as the ‘pain signal’ for simplicity. However, the signal A also can include any number of sensory and motor sub-signals; a selected sensory sub-signal being the pain signal. In other words, pain is just one of many signals traveling along a certain nerve 105. Unfortunately, pain is not easily recognized amongst the multitude of other sensory information. In order to keep side-effects like numbness to a minimum, it is desirable to narrow the cancellation effect close to the actual pain signal and leave as many of the other signals as possible unaffected.

Accordingly, to minimize side-effects, the rPU 107 can transmit a 2-dimensional mapping of all nerve signals included in the signal A to the mobile device 113 (e.g., shown in FIGS. 8B-D).

Advantageously, the patient can be in control of trimming the cancellation effect as closely as possible around the original pain signal A by narrowing, widening, and moving the cancellation signal B window on the mobile device 113 (also discussed with reference to FIGS. 8A-D). The goal is, to narrow the cancellation signal B as much as possible, while still not experiencing any pain.

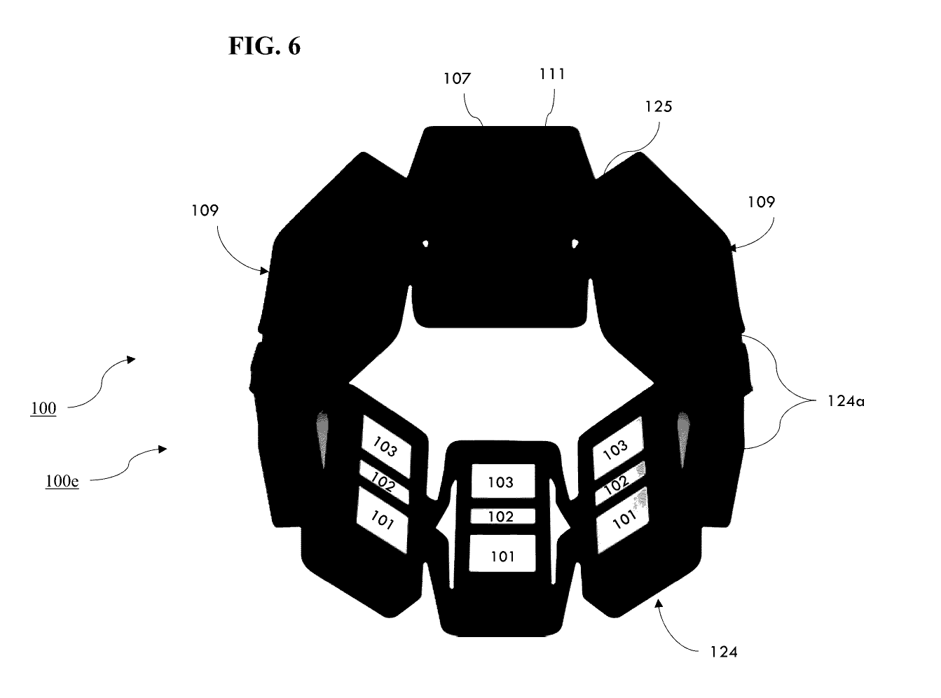

Turning to FIG. 6, an exemplary design of the pain reduction system 100c is shown. The pain reduction system 100c includes a housing 124. The housing 124 includes one or more housing compartments 124a which can be mechanically connected by flexible hinged means 125 to allow conforming placement of the housing 124 on a curved portion of the human anatomy. A selected housing compartment 124a can house the rPU 107 and the wireless transmitter 111; one or more housing compartments 124a provide housing for the batteries 109; one or more housing compartments 124a can provide housing (or carrying via detachable means) for the sensors 101, 103 and the pulse generator 102.

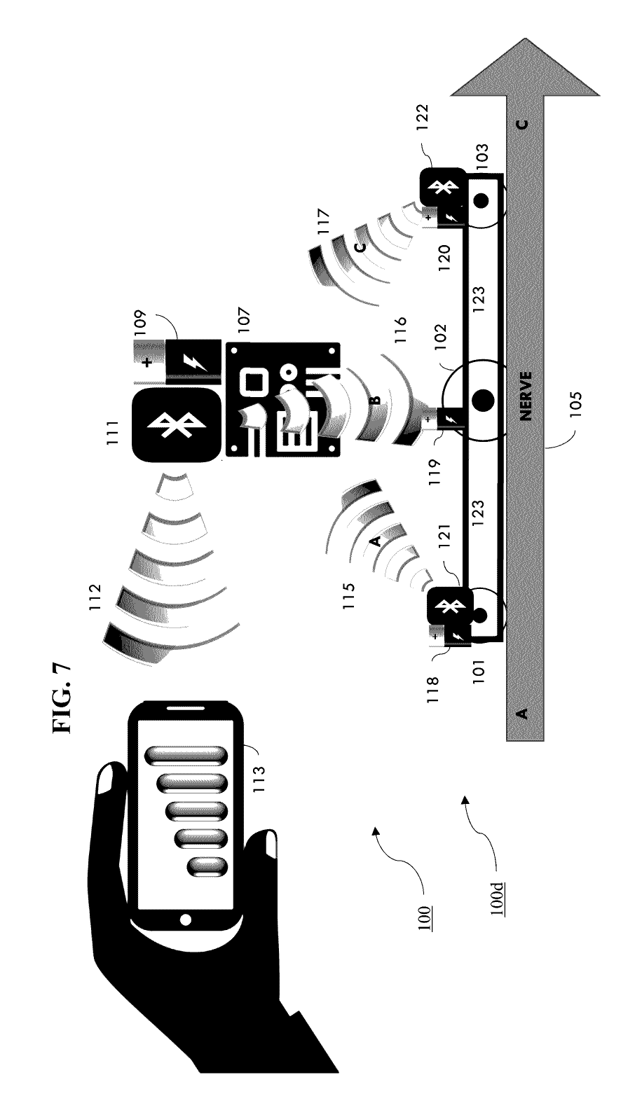

Although the previous embodiments included wired connections between the topical sensors (e.g., the sensors 101 and 103), the pain reduction system 100 can also include wireless connectivity to provide additional versatility. Turning now to FIG. 7, another embodiment of the pain reduction system 100 is shown. As shown in FIG. 7, a pain reduction system 100d includes the topical sensors 101, 103 and the pulse generator 102 on a carrier medium 123 (for example, but not limited to, a plastic foil). The carrier medium 123 also includes several wireless transmitters (e.g., carrier wireless transmitters 121 and 122) in order to connect the sensors 101 and 103 wirelessly to the rPU 107. In some embodiments, the pain reduction system 100c can include additional batteries 118, 119, and 120 to power the sensors 101, 103, the pulse generator 102, and the wireless transmitters 121, 122. In some embodiments, the additional batteries 118, 119, and 120 are unique from one another. In some embodiments, the additional batteries 118, 119, and 120 is a single unit that powers all units 101, 103, 102, 121, and 122 via the carrier medium 123 that can be conductive. A sensor, pulse generator, and wireless transmitter (collectively referred to as sensor array) separate from the rPU 107 is created, therefore, allowing the patient to place a much lighter and smaller sensor array on the skin while carrying the heavier rPU 107 (together with wireless transmitter 111 and the battery 109) conveniently in a pocket or anywhere in wireless range.

The topical sensor 101 reads the pain signal A traveling through the nerve 105 and transmits this signal to the reLeaph processing unit (rPU) 107 via the wireless transmitter 121 and a wireless connection 115 (for example, but not limited to, a Bluetooth connection). In order for the sensor 101 to successfully read nerve signal A, the sensor 101 can be positioned right on top of the nerve 105.

In some embodiments, the pain reduction system 100d can operate in a positioning mode via a mobile application on a mobile device 113 (e.g., smartphone) which is connected to the rPU 107 via the wireless connection 112 (e.g., such as via Bluetooth) and the wireless transmitter 111. As shown, the mobile device 113 is connected via the wireless connection 112, thereby reducing the number of wires and maintaining a convenient handling by a user; however, it should be understood that a wired connection also is within the scope of this embodiment. The sensor 101 can be moved across the skin in roughly the vicinity of the nerve 105. The mobile device 113 shows the nerve signal strength and the sensor array can be positioned such that the sensor 101 is positioned where the highest signal level is shown.

The rPU 107 creates an opposing cancellation signal B and transmits this signal via the wireless transmitter 111 and a connection 116 (for example, but not limited to, a Bluetooth connection) to pulse generator 102. This pulse generator 102 is positioned topically and close to the sensor 101 (e.g., about three centimeters from the sensor 101 and farther away from the pain afflicted area and closer to the pain receptors than the position of the sensor 101). In a preferred embodiment, the original pain signal A, still traveling along the nerve 105, and the cancellation signal B cancel each other out once they collide.

In this embodiment, the rPU 107 calibrates for nerve velocity automatically. In an automatic calibration mode, the rPU 107 emits a test signal through the pulse generator 102, measures the time until this test signal reaches the sensors 101, 103 and determines pertaining nerve velocity. According to this value, the rPU 107 delays and/or advances the cancellation signal B by a predetermined time to match it with the initial pain signal A.

In some embodiments, the rPU 107 can automatically calibrate for weakened and distorted signals, such as explained with reference to FIG. 14.

The sensor 103, positioned in close neighborhood (e.g., within 3 centimeters) of the pulse generator 102 (e.g., farther away from the pain afflicted area and closer to the pain receptors than the pulse generator 102), reads the resulting collision signal C and sends it back to the rPU 107 via the wireless transmitter 122 and the wireless connection 117. The rPU 107 determines a delta (Δ) between an optimum collision result and the actual collision signal C and corrects subsequent cancellation signals B for Δ in order to converge to the optimum collision result: a cancelled pain signal and a patient free of pain.

Again, to reduce unnecessary side-effects, the rPU 107 can transmit a 2-dimensional mapping of all nerve signals contained in the pain signal A to the mobile device 113.

Advantageously, the patient can be in control of trimming the cancellation effect as closely as possible around the original pain signal A by narrowing, widening, and moving the cancellation signal B window on the mobile device 113. The goal is, to narrow the cancellation signal B as much as possible, while still not experiencing any pain.

The pain reduction system 100 analyzes the waveform of the nerve signal 105, then generates a signal that will invert the polarity of the original signal. This inverted signal (in antiphase) is then amplified and a transducer creates a wave directly proportional to the amplitude of the original waveform, utilizing destructive interference. This effectively eliminates the original nerve signal.

Turning to FIG. 8A, waves influence each other when they get close or collide; exactly opposite waves even cancel each other out. In physics this phenomenon in which two waves superpose to form a resultant wave of greater or lower amplitude is called interference.

The principle of superposition of waves states that when two or more propagating waves of the same type are incident on the same point, the total displacement at that point is equal to the sum of the displacements of the individual waves. If a crest of a wave meets a crest of another wave of the same frequency at the same point, then the magnitude of the displacement is the sum of the individual magnitudes—this is constructive interference as shown on the left of FIG. 8A. If a crest of one wave meets a trough of another wave, then the magnitude of the displacements is equal to the difference in the individual magnitudes—this is known as destructive interference as shown on the right of FIG. 8A.

A common application of this principle is noise cancellation: Sound is a pressure wave, which includes alternating periods of compression and rarefaction. A noise-cancellation speaker emits a sound wave with the same amplitude but with inverted phase (also known as antiphase) to the original sound. The waves combine to form a new wave in the above described interference process and effectively cancel each other out.

Turning to FIG. 8B, an exemplary electromagnetic wave is shown to represent a complete spectrum of signals recorded from a nerve cell 105. This electromagnetic wave can, for example, represent the pain signal A that is detected by the pain reduction system 100. With reference to FIG. 8C, a corresponding cancellation signal B is shown. For example, the pain reduction system 100 receives the pain signal A shown in FIG. 8B and generates a phase shifted cancellation signal B such that a crest of the pain signal A meets a trough of the wave signal B. Accordingly, when the pulse generator 102 emits the cancellation signal B, destructive interference of the two signals can interrupt the pain signal A before it can reach the human brain.

In some embodiments, and with reference to FIG. 8D, a complete interruption of signal transmission will be undesirable and only a certain bandwidth of signals (reflecting the actual pain) is to be eliminated. The remaining nerve signal is untouched to minimize side-effects (e.g., unnecessary numbness and involuntary tremors). Accordingly, FIG. 8D illustrates an exemplary bandwidth of signals that can be eliminated by the pain reduction system 100 to reduce any undesired side-effects.

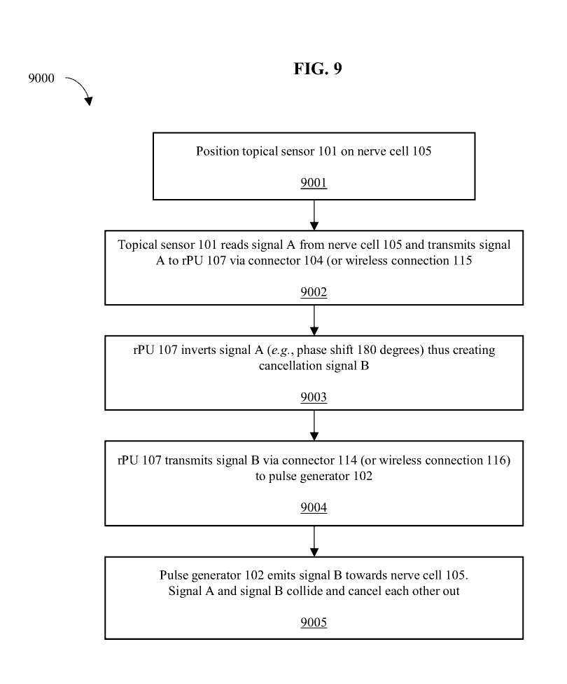

The pain reduction system 100 can reduce pain in any suitable manner discussed above, such as a pain reduction process 9000 as shown in FIG. 9. Turning to FIG. 9, the pain reduction process 9000 begins when the topical sensor 101 is positioned on or near the nerve cell 105, at 9001. The topical sensor 101 reads the original pain signal A from the nerve cell 105 and relays the signal A to the rPU 107 via the connector 104 (or the wireless transmitter 121 and the wireless connection 115), at 9002. Once the original pain signal A has been received, the rPU 107 generates the cancellation signal B by inverting the original pain signal A, at 9003. For example, the rPU 107 can receive the original pain signal A and perform a phase shift by one hundred eighty degrees (180°) to generate the cancellation signal B.

The rPU 107 transmits the cancellation signal B via the connector 104 (or the wireless transmitter 111 and the wireless connection 116 to the pulse generator 102, at 9004. The pulse generator 102 emits signal B towards nerve cell 105 creating a collision between original signal A and cancellation signal B, at 9005. In a preferred embodiment, the resulting collision signal C is not transmitting any pain. Factors that can affect the collusions signal C and a method for adjusting these factors are discussed, for example, with reference to FIG. 14.

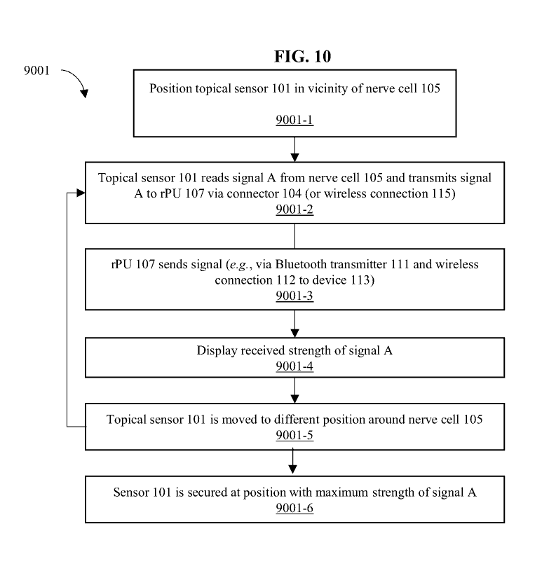

Now turning to FIG. 10, one embodiment for positioning the topical sensor on the nerve cell 105 (i.e., step 9001) in relation to the embodiments displayed in FIGS. 3, 4, 5, and 7 is shown in further detail. Initially, the topical sensor 101 is placed in a close vicinity of the nerve 105. For example, the topical sensor 101 is positioned roughly within a 4 square centimeter area), at 9001-1. The topical sensor 101 reads the signal A from the nerve cell 105, at 9001-2. At this point the pain reduction system 100 determines the signal strength of the pain signal A and transmits the pain signal A to the rPU 107 via the connector 104 (or the wireless transmitter 121 and the wireless connector 115), at 9001-2. The rPU 107 transmits the pain signal A via the wireless transmitter 111 and the wireless connection 112 (or in some cases an equivalent wired connection) to the mobile device 113, at9001-3, where a visual approximation of the received signal strength is displayed, at 9001-4. In order to determine the ideal position for sensor 101 where the highest signal strength is received (closest to the nerve cell 105), the sensor 101 can be repositioned, at 9001-5, and the above process (steps 9001-2,9001-3,9001-4, and 9001-5) repeats until the ideal position is determined and the sensor 101 is properly positioned, at 9001-6.

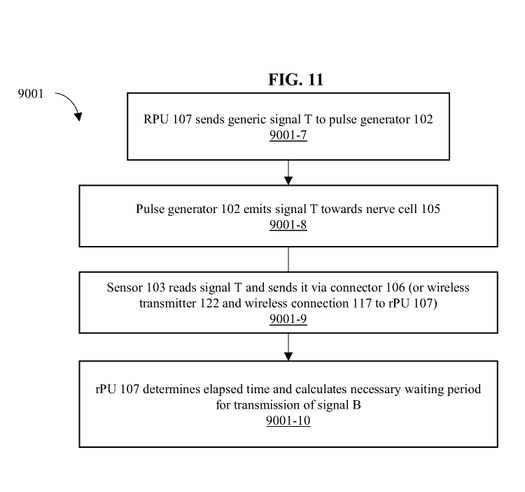

With reference now to FIG. 11, another embodiment for positioning the topical sensor on the nerve cell 105 (i.e., step 9001) is shown as including a determination of the velocity that the pain signal A travels along the nerve cell 105. As nerve signals are relatively slow, determination of the velocity allows the pulse generator 102 to emit the cancellation signal B to nerve cell 105 at a predetermined time in order to create a collision. For example, if the pulse generator 102 transmits the pain signal A early, the cancellation signal B will not interfere and the pain signal A is not effected.

In one embodiment, the rPU 107 provides a generic, but unique, signal T to the pulse generator 102. The pulse generator 102 emits the signal T towards the nerve cell 105; when the signal T reaches the nerve cell 105, the signal T will travel along the nerve cell 105 towards the test sensor 103. When the signal T reaches the sensor 103, the sensor 103 receives the signal T and sends it via the connector 106 (or the wireless transmitter 122 and the wireless connector 117) to the rPU 107. The rPU 107 determines the elapsed time from sending the signal T through the pulse generator 102 to receiving the signal T through the test sensor 103. This elapsed time is used to calculate any desired waiting period (delay) for subsequent transmissions of the cancellation signal B.

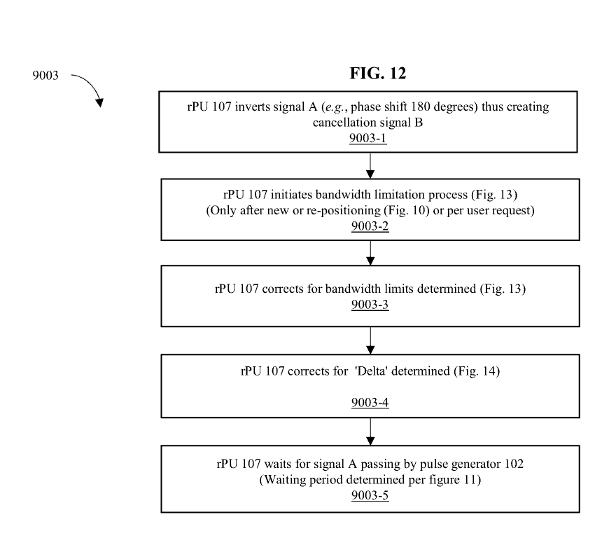

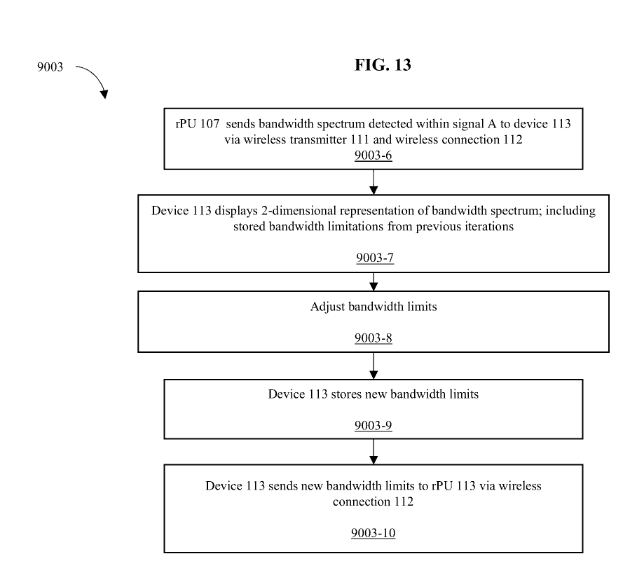

Turning now to FIG. 12, one embodiment for generating the cancellation signal B (step 9003) is illustrated in further detail. In one embodiment, the rPU 107 generates the cancellation signal B by inverting the pain signal A (e.g., phase shift 180 degrees). The embodiment shown in FIG. 9 also provides for dampening effects caused by a patient's tissue, to allow for transmission velocity of nerve cell 105, and to eliminate unwanted side effects like numbness or tremors. After creating the ‘raw’ signal B (at 9003-1), the rPU 107 initiates a bandwidth limitation process, at 9003-2). As previously discussed, the pain signal A includes many different signals; only one of them is related to a patient's pain experience. In case the ‘raw’ signal B is sent back to the nerve cell 105, the resulting collision cancels out not only the pain signal but all other motor and sensory signals that are part of signal A as well. While the main objective—eliminating a patient's pain—is accomplished, a multitude of unwanted and maybe bothersome side effects can be experienced by the patient. The bandwidth limitation process (step 9003-2) reduces/eliminates these side effects and will be further described with reference to FIG. 13. Note that band limitation process. 9003-can be invoked after an initial positioning process (shown in FIG. 10) or per user request and is only invoked until the user decides that the bandwidth limits are optimal. In the next step (9003-3), the rPU 107 alters the signal B according to the bandwidth limits determined by the rPU 107 in step 9003-2. The rPU 107 corrects the signal B for tissue dampening effects determined by the process further described in FIG. 14. In step 9003-5, the rPU 107 waits for signal A to pass the pulse generator 102. The waiting period can be determined by the process described in FIG. 11.

Turning to FIG. 13, one embodiment for determining the bandwidth limitations for signal B is shown; thus eliminating unwanted side effects like tremors and numbness. Receiving the pain signal A from the sensor 101, the rPU 107 determines the bandwidth spectrum of signal A (shown in FIG. 8D) and sends the upper and lower limit of the spectrum to the mobile device 113 via the wireless transmitter 111 and wireless connection 112, at 9003-6. The mobile device 113 can display a visual representation of the bandwidth spectrum to the user (including already stored bandwidth limits previously determined by the user) (at 9003-7). In step 9003-8, the user adjusts the bandwidth limits on the mobile device 113. In step 9003-9, the mobile device 113 stores the new bandwidth limit values and in step 9003-10, and the mobile device 113 sends the new bandwidth limits to the rPU 107 via the wireless connection 112.

Now turning to FIG. 14, one embodiment for determining the correction value for dampening effects caused by a patient's tissue is shown. The sensor 103 reads the collision signal C from the nerve cell 105. The collision signal C is the result of the collision between original signal A and cancellation signal B. This method, can be non-invasive, advantageously allowing all measurements to be taken topically. The tissue between the nerve cell 105 and the sensor 101 and the pulse generator 102 influences the quality and especially the intensity of readings and also the intensity of the cancellation signal B while it is trying to reach nerve cell 105 through the tissue. Therefore, the collision signal C does not really represent the collision of signal A and signal B, but the collision of weakened signal A and B. Therefore, without further correction the collision between signal A and B would not result in the ideal 0-amplitude collision signal. In step 9003-12 rPU 107 determines the ‘delta’ between the expected and wanted signal C and the real reading of signal C. In step 9003-13 rPU 107 calculates a correction value for signal B in order to achieve the ideal result in future collisions.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives.

What is claimed is:

1. A method for pain reduction, the method comprising:
receiving a pain signal from a topical sensor positioned on a nerve cell;
generating a cancellation signal based on the received pain signal via a processing unit; and
emitting the cancellation signal towards the nerve cell through a pulse generator.

2. The method of claim 1, wherein said generating the cancellation signal comprises inverting the pain signal.

3. The method of claim 2, wherein said inverting the pain signal includes performing a phase shift of the pain signal by one hundred eighty degrees.

4. The method of claim 3, wherein said emitting the cancellation signal provides a destructive interference of the pain signal and the cancellation signal.

5. The method of claim 1, further comprising determining a position on the nerve cell that provides a highest magnitude of the pain signal.

6. The method of claim 5, further comprising positioning a selected topical sensor at the determined position of the nerve cell.

7. The method of claim 1, further comprising determining a nerve velocity based on the received pain signal, wherein said emitting is based on the determined nerve velocity.

8. A system for pain reduction, the system comprising:
one or more topical sensors for placement on a nerve cell for receiving a pain signal;
a processing unit in communication with said one or more topical sensors for generating a cancellation signal based on the received pain signal; and
a pulse generator positioned on the nerve cell for receiving a control signal from said processing unit and for emitting the cancellation signal towards the nerve cell, wherein the emitted cancellation signal destructively interferes with the pain signal.

9. The system of claim 8, wherein said processing unit generates the cancellation signal by inverting the received pain signal.

10. The system of claim 8, further comprising a test sensor in communication with said processing unit and positioned on the nerve cell at a location unique from the placement of the pulse generator for transmitting a test signal and determining a nerve velocity.

11. The system of claim 10, wherein said processing unit provides the control signal to the pulse generator for emitting the cancelling signal based on the determined nerve velocity.

12. The system of claim 8, further comprising a mobile device in communication with said processing unit for displaying the received pain signal and for providing the control signal to said pulse generator.

13. The system of claim 12, wherein said mobile device is in wireless communication with said processing unit.

14. The system of claim 8, wherein said processing unit is a reLeaph processing unit.

15. The system of claim 8, wherein said processing unit is in wireless communication with said topical sensors.

16. The system of claim 8, further comprising a housing that includes one or more housing components, each housing component flexibly hinged to another housing component to allow placement of the housing on a curved portion of a patient; each housing component containing at least one of said processing unit, a selected topical sensor, and said pulse generator.

17. A method for pain reduction, the method comprising:
receiving a pain signal from a topical sensor positioned on a nerve cell;
generating a cancellation signal based on the received pain signal via a processing unit;
transmitting a test signal into the nerve cell through a pulse generator in communication with the processing unit;
receiving the transmitted test signal via one or more test sensors in communication with the processing unit;
determining a nerve velocity of the nerve cell based on the received test signal; and
emitting the cancellation signal towards the nerve cell through a pulse generator based on the determined nerve velocity for providing destructive interference of the pain signal.

18. The method of claim 17, wherein said generating the cancellation signal comprises performing a phase shift of the pain signal by one hundred eighty degrees.

19. The method of claim 18, wherein said emitting the cancellation signal provides a destructive interference of the pain signal and the cancellation signal.

20. The method of claim 17, further comprising:
determining a position on the nerve cell that provides a highest magnitude of the pain signal; and
positioning a selected topical sensor at the determined position of the nerve cell.
